(19) United States
(12) Patent Application Publication
(10) Pub. No.: US 2024/0110916 A1
(43) Pub. Date: Apr. 4, 2024

(54) METHOD FOR TREATING HEPATOCELLULAR CARCINOMA

(71) Applicant: Academia Sinica, Taipei City (TW)

(72) Inventors: Yun-Ru CHEN, Taipei City (TW); Jin-Lin WU, Taichung City (TW); Pei-Jer CHEN, New Taipei City (TW); Tung-Hung SU, Taipei City (TW)

(73) Assignee: Academia Sinica, Taipei City (TW)

(21) Appl. No.: 18/273,762

(22) PCT Filed: Jan. 21, 2022
(86) PCT No.: PCT/US2022/013211

§ 371 Date: Jul. 22, 2023

Related U.S. Application Data
(60) Provisional application No. 63/140,807, filed on Jan. 23, 2021.

(51) Int. Cl. G01N 33/574; A61K 45/06; G01N 1/28; G01N 33/68

(52) U.S. Cl. CPC G01N 33/57438; A61K 45/06; G01N 1/28; G01N 33/6893; G01N 2333/471; G01N 2333/775

(57) ABSTRACT

Disclosed herein is a method for identifying and treating an early-stage hepatocellular carcinoma (HCC) in a subject. The method mainly includes determining the level of serum amyloid A (SAA) protein, and providing anti-cancer treatment based on the determined level of SAA protein. According to some embodiments of the present disclosure, the anti-cancer treatment is provided when the determined level of SAA protein is lower than that of a first control sample, or when the determined level of SAA protein is higher than that of a second control sample. In some embodiments, the first control sample is derived from a subject having a late stage HCC, and the second control sample is derived from a subject having a liver disease that is any of hepatitis, liver cirrhosis, or a combination thereof.

### CROSS-REFERENCES TO RELATED APPLICATIONS

This application is a U.S. National Stage Filing under 35 U.S.C. 371 § from International Patent Application Serial No. PCT/US22/13211, entitled “METHOD FOR TREATING HEPATOCELLULAR CARCINOMA,” filed on Jan. 21, 2022, and published on Jul. 28, 2022, which claims the priority and the benefit of U.S. Provisional Patent Application No. 63/140,807, filed Jan. 23, 2021, the diclosure of which are incorporated by reference herein in their entireties.

### BACKGROUND OF THE INVENTION

### Field of the Invention

The present disclosure relates to disease identification and treatment. More particularly, the disclosure invention relates to a method of identifying and treating an early-stage hepatocellular carcinoma (HCC).

### Description of Related Art

Hepatocellular carcinoma (HCC) is the most common primary liver malignancy and the third most frequent cause of cancer-related death worldwide. The incidence of HCC depends on variable prevalence of chronic hepatitis B virus (HBV) and hepatitis C virus (HCV) infection. The Barcelona Clinic Liver Cancer (BCLC) system classifies HCC into five stages, stage 0, A, B, C, and D, based on the number and the size of tumors, liver reserve, and the overall performance status of the patients. Early diagnosis and treatment improve survival rate in HCC, however, current curative methods for HCC can be applied to patients only at early disease stages; treatment options for advanced HCC are non-curative.

Though current curative therapies including radiofrequency ablation, surgical resection, and orthotopic liver transplantation are only feasible for patients with early-stage HCC, early detection for HCC has many limitations. Specifically, early HCC detection is achieved by imaging examination and serological tests. However, the surveillance by abdominal ultrasonography is limited by its image quality and strongly relied on the experience of the operator. Technologies with higher diagnostic accuracy such as computed tomography (CT) and magnetic resonance imaging (MRI) are not widely utilized due to high cost and low accessibility.

Alternatively, serological biomarkers for HCC surveillance including α-fetoprotein (AFP), lectin-reactive AFP (AFP-L3), and prothrombin induced by vitamin K absence II (PIVKA II)/des-gamma-carboxy prothrombin (DCP) are widely used in HCC detection; however, all these biomarkers alone demonstrated low sensitivity for early HCC diagnosis. Combination of AFP and AFP-L3 showed good sensitivity to early HCC, but there are still 15-30% of advanced HCC patients whose serum AFP level remains normal. None of said biomarkers can make highly reliable diagnosis for early HCC.

In view of the foregoing, there exists in the related art a need of a novel diagnosis and treatment of early-stage HCC.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a method of identifying and treating a subject having an early-stage hepatocellular carcinoma (HCC). The method comprises steps of: (a) obtaining a biological sample from the subject; (b) determining the level of serum amyloid A (SAA) protein in the biological sample; and (c) providing an anti-cancer treatment to the subject when the determined level of SAA protein is lower than that of a first control sample, or higher than that of a second control sample. In the step (c) of present method, the anticancer treatment is selected from the group consisting of a surgery, a radiofrequency ablation, a laser ablation, a microwave ablation, a systemic chemotherapy, a percutaneous alcohol injection, a percutaneous acetic acid injection, a transarterial chemoembolization, an adjuvant immunotherapy, an adjuvant antiviral treatment, an orthotopic liver transplantation, and a combination thereof.

According to some embodiments of the present disclosure, the first control sample is derived from a subject having a late stage HCC; and the second control sample is derived from a subject having a liver disease that is any of hepatitis, liver cirrhosis, or a combination thereof.

According to some embodiments of the present disclosure, the SAA protein is in monomeric form.

According to one embodiment of the present disclosure, the level of SAA protein in the first control sample is at least 0.9 ng; and the level of SAA protein in the second control sample is no greater than 0.9 ng or no greater than 180 ng/mL.

According to one embodiment of the present disclosure, the subject has a serum alpha-fetoprotein (AFP) level lower than 10 ng/mL.

Examples of the biological sample suitable for use in the present disclosure include, but are not limited to, a whole blood sample, a serum sample, a plasma sample, a urine sample, a saliva sample, a cerebrospinal fluid sample, a synovial fluid sample, a pleural fluid sample, a peritoneal fluid sample, and a lymph sample. According to one working example, the biological sample is a serum sample.

Another aspect of the present disclosure is directed to a method of identifying and treating a subject having an early-stage HCC. The method comprises steps of: (a) obtaining a biological sample from the subject; (b) subjecting the biological sample to an ultrasonic treatment; (c) determining the level of serum amyloid A (SAA) protein in the ultrasonic treated biological sample of step (b); and (d) providing an anti-cancer treatment to the subject when the determined level of SAA protein is higher than that of a control sample.

According to some embodiments of the present disclosure, the anticancer treatment is selected from the group consisting of a surgery, a radiofrequency ablation, a laser ablation, a microwave ablation, a systemic chemotherapy, a percutaneous alcohol injection, a percutaneous acetic acid injection, a transarterial chemoembolization, an adjuvant immunotherapy, an adjuvant antiviral treatment, an orthotopic liver transplantation, and a combination thereof, and the control sample is derived from a subject having a liver disease that is any of a late stage HCC, hepatitis, cirrhosis, or a combination thereof.

According to one embodiment of the present disclosure, the subject has a serum alpha-fetoprotein (AFP) level lower than 10 ng/mL. According to a preferred embodiment, the subject has the AFP level lower than 7 ng/mL.

Examples of the biological sample suitable for use in the present method include, but are not limited to, a whole blood sample, a serum sample, a plasma sample, a urine sample, a saliva sample, a cerebrospinal fluid sample, a synovial fluid sample, a pleural fluid sample, a peritoneal fluid sample, and a lymph sample. According to one working example, the biological sample is a serum sample.

Still another aspect of the present disclosure is directed to a method of diagnosing an early-stage HCC in a subject via use of a biological sample isolated from the subject. The method comprises determining the level of serum amyloid A (SAA) protein in the isolated biological sample, wherein a difference between the determined level of SAA in the isolated biological sample and that of a control sample indicates that the subject has the early-stage HCC; and the control sample is derived from a subject having a liver disease that is any of a late stage HCC, hepatitis, cirrhosis, or a combination thereof.

According to some embodiments of the present disclosure, the SAA protein is in monomeric form.

According to one alternative embodiment of the present disclosure, the method further comprises subjecting the isolated biological sample to an ultrasonic treatment before determining the level of SAA protein therein.

According to one embodiment of the present disclosure, the subject has a serum alpha-fetoprotein (AFP) level lower than 10 ng/mL. According to a preferred embodiment, the subject has the AFP level lower than 7 ng/mL.

Examples of the isolated biological sample suitable for use in the present disclosure include, but are not limited to, a whole blood sample, a serum sample, a plasma sample, a urine sample, a saliva sample, a cerebrospinal fluid sample, a synovial fluid sample, a pleural fluid sample, a peritoneal fluid sample, and a lymph sample. According to one working example, the isolated biological sample is a serum sample.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIGS. 1A-1C respectively depict the results of the assessment of total A-SAA level in sera of the hepatitis, LC, and HCC cohorts. The concentration of total A-SAA in serum was detected using ELISA. A total of 166 serum samples from 40 hepatitis (indicated as H in all figures), 30 LC, and 96 HCC patients were measured and analyzed. FIG. 1A indicates serum A-SAA levels in H, LC, and HCC cohorts. Area under the receiver operating characteristic (AUROC) curves for discrimination of H vs. HCC and LC vs. HCC are respectively depicted in FIG. 1B and FIG. 1C. The statistical analysis was performed by one-way ANOVA and Tukey's Post Hoc Test, where p<0.01 (**) and <0.001 (***).

FIGS. 2A-2C respectively depict the results of the amount of monomeric A-SAA among three cohorts and its diagnostic performance for HCC. Hepatitis is indicated as H. FIG. 2A depicts the quantified level of monomeric A-SAA in H, LC, and HCC cohorts. AUROC curves of monomeric A-SAA for discrimination of H vs. HCC and LC vs. HCC are respectively depicted in FIG. 2B and FIG. 2C. The statistical analysis for comparison among different cohorts was performed by one-way ANOVA and Tukey's Post Hoc Test, where p<0.05 (*), <0.01 (**) and p<0.0001 (****).

FIGS. 3A-3C respectively depict the results of the level of monomeric A-SAA in the progression of HCC. The quantification of the amount of monomeric A-SAA from Western blot results was conducted using image analysis software. The amount of monomeric A-SAA at different BCLC stages were depicted in FIG. 3A and FIG. 3B; and AUROC curve of monomeric A-SAA for early-stage vs. advanced stage was depicted in FIG. 3C. The statistical analysis for comparison among different cohorts was performed by one-way ANOVA and Tukey's Post Hoc Test, where p<0.05 (*) and p<0.01 (**). The statistical analysis for comparison between early and advanced stage was conducted by Mann-Whitney U test, where p<0.0001 (****).

FIGS. 4A-4F respectively depict the results of the evaluation of SAA protein as a potential biomarker for detecting HCC. The signal amplification of serum A-SAA was conducted by PMCA. A total of 146 serum samples from 30 hepatitis (indicated as H), 30 LC, and 86 HCC patients were measured and analyzed. Normalized ThT signals after PMCA in H, LC, and HCC cohorts were depicted in FIG. 4A. FIG. 4B further depicts the result of AUROC curves for LC vs. HCC. The statistical analysis was performed by one-way ANOVA and Tukey's Post Hoc Test, where p<0.05 (*), p<0.0001 (****). FIG. 4C indicates a comparison of the normalized ThT signal between LC and early-stage HCC, and the statistical analysis was conducted by Mann-Whitney U test, where p<0.0001 (****). FIG. 4D depicts the AUROC curve for discrimination between LC and early-stage of HCC. FIG. 4E depicts the AUROC curve for discrimination between LC and stage 0 of HCC. FIG. 4F depicts the AUROC curve for discrimination between LC and stage A of HCC.

FIGS. 5A-5C respectively depict the results of HCC diagnostic capacity of monomeric A-SAA in a low AFP level. FIG. 5A depicts the amount of monomeric A-SAA in H, LC, and HCC cohorts with low AFP levels. The statistical analysis was performed by one-way ANOVA and Tukey's Post Hoc Test, where p<0.001 (***). In FIG. 5B and FIG. 5C, AUROC curves of the amount of monomeric A-SAA and their AUCs for H vs. HCC and LC vs. HCC are respectively shown.

FIGS. 6A-6D respectively depict the results of HCC diagnostic capacity of PMCA in low AFP level. FIG. 6A depicts the normalized ThT signals after PMCA in H, LC, and HCC cohorts. FIG. 6B depicts the AUROC curves of PMCA results and their AUCs for H vs. HCC (left panel) and LC vs. HCC (right panel). FIG. 6C indicates the comparison of the normalized ThT signal between LC and early-stage HCC. The statistical analysis was conducted by Mann-Whitney U test, where p<0.0001 (****). FIG. 6D, AUROC curve of PMCA results for LC vs. early-stage HCC.

FIGS. 7A-7C respectively depict the results of HCC diagnostic capacity of PMCA in patients with cirrhosis in liver. FIG. 7A depicts the normalized ThT signals after PMCA in LC, and early, intermediate, and advanced stages of HCC. The statistical analysis was performed by one-way ANOVA and Tukey's Post Hoc Test, where p<0.05 (*), p<0.001 (***), and p<0.0001 (****). FIGS. 7B and 7C respectively depict the AUROC curves of PMCA signals and their AUCs for discrimination between cirrhotic liver vs. all-stage HCC, and cirrhotic liver vs. early-stage of HCC.

FIGS. 8A-8B respectively depict the results of PMCA signals in discrimination of all-stage and early-stage HCC in cirrhotic patients with low AFP level. FIG. 8A depicts an AUROC curve of PMCA method for discrimination between cirrhotic liver and all-stage HCC; and FIG. 8B depicts an AUROC curve of PMCA method for discrimination between cirrhotic liver and early-stage HCC.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example.

However, the same or equivalent functions and sequences may be accomplished by different examples.

### Definitions

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

The Barcelona Clinic Liver Cancer (BCLC) staging system classifies the course of hepatocellular carcinoma into five stages, which are Stage 0, Stage A, Stage B, Stage C, and Stage D, based on the number and the size of tumors, liver reserve, and the overall performance status of a patient. According to the definition in the BCLC staging system, in Stage 0, the single nodule is less than 2 cm, the liver remains normal function, and the patient would not be aware of abnormalities. Once a single tumor of any size, or up to 3 tumors become less than 3 cm, then it comes to Stage A. Normally in Stage A, the liver retains normal functions. The term “early-stage hepatocellular carcinoma (HCC)” used herein the present application refers to Stage 0 and/or Stage A. The term “late stage HCC” is relative to said term “early-stage HCC”, the former refers to Stage B, C, and D, unless indicated otherwise. In the late stage HCC, the liver is multinodular and liver function begins to decline.

The term “biological sample” refers to any sample including tissue samples (such as tissue sections and needle biopsies of a tissue); cell samples (e.g., cytological smears (such as Pap or blood smears) or samples of cells obtained by microdissection); or cell fractions, fragments or organelles (such as obtained by lysing cells and separating the components thereof by centrifugation or otherwise). Other examples of biological samples include whole blood, serum, plasma, urine, saliva, cerebrospinal fluid, synovial fluid, pleural fluid, peritoneal fluid, lymph, or a combination thereof.

The term “level of protein” refers to a quantitative number that is capable of indicating, showing, stating, directly mentioning, expressing the amount of protein production (e.g., serum amyloid A (SAA) protein or acute-phase SAA (A-SAA) protein) within certain condition, regardless which unit is used. Unless indicated otherwise, “level of protein” is quantified and indicated by the units well known in the art, which includes, but are not limited to, a concentration, a volume, a number, a molecular mass, and etc. According to the present disclosure, the level of SAA or A-SAA protein is indicated by a concentration or a normalized number in volume.

The term of “acute-phase” protein used herein refers to a class of proteins whose plasma concentrations increase (positive acute-phase proteins) or decrease (negative acute-phase proteins) in response to inflammation. This response is called the acute-phase reaction (also called acute-phase response). Serum amyloid A (SAA) protein family has four members, SAA1 to 4, and all of them are positive acute-phase reactants produced by hepatocytes. Among four family members, SAA1 and SAA2 share high sequence identity and have significant increased expression during an acute-phase response, and are collectively referred as “acute-phase serum amyloid A (A-SAA)” in the present methods and studies.

The term “PMCA signal” used herein refers to a quantified signal of the present SAA protein (i.e., monomeric and/or oligomeric forms of A-SAA) after conducting protein misfolding cyclic amplification (PMCA), followed by normalization with the aid of fluorescent dyes (e.g., Thioflavin), in which the fluorescent intensity of the fluorescent dye bound to A-SAA protein was determined. According to embodiments of the present disclosure, the PMCA signal of A-SAA protein serves as a biomarker indicative of liver carcinoma; in one preferred embodiment, the PMCA signal is indicative of early-stage of HCC.

The term “treating” or “treatment” encompasses partially or completely preventing, ameliorating, mitigating and/or managing a symptom, a secondary disorder or a condition associated with a disease, for example, an early-stage hepatocellular carcinoma (HCC). The term “treating” or “treatment” as used herein refers to application or administration of one or more therapeutic agent, ablation, immunoadjuvant or surgery to a subject, who has a symptom, a secondary disorder or a condition associated with a disease, with the purpose to partially or completely alleviate, ameliorate, relieve, delay onset of, inhibit progression of, reduce severity of, and/or reduce incidence of one or more symptoms, secondary disorders or features associated with the disease. Treatment may be administered to a subject who exhibits only early signs of, or has a certain level of indicators (e.g., a biomarker) linked to, such symptoms, disorder, and/or condition for the purpose of decreasing the risk of developing the symptoms, secondary disorders, and/or conditions associated with a disease. Treatment is generally “effective” if one or more symptoms or clinical markers are reduced as that term is defined herein. Alternatively, a treatment is “effective” if the progression of a symptom, disorder or condition is reduced or halted.

The term “diagnosing” refers to methods by which the skilled artisan can estimate and/or determine the probability (“a likelihood”) of whether or not a patient is suffering from a given disease or condition. That such a diagnosis is “determined” is not meant to imply that the diagnosis is 100% accurate, but to provide a reliable result with the area under the receiver operating characteristic (AUROC) at least 0.7. Many biomarkers are indicative of multiple conditions. The skilled clinician either use biomarker results in an informational vacuum or use them together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

The term “subject” or “patient” is used interchangeably herein and is intended to mean a mammal including the human species that is treatable by the method of the present disclosure. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated.

### DETAIL DESCRIPTION OF PREFERRED EMBODIMENTS

The present disclosure is based, at least in part, on the discovery that the amount of serum amyloid A (SAA) protein, both the monomeric form and the oligomeric form, are significantly higher in hepatocellular carcinoma (HCC) patients. In addition, the level of monomeric SAA protein is capable of differentiating early-stage HCC from late stage HCC, or from other hepatic diseases such as hepatitis (H) and liver cirrhosis (LC), even in low serum alpha-fetoprotein (AFP) patients, therefore may be used to facilitate the early diagnosis and treatment of HCC. Accordingly, the present disclosure aims at providing a method of identifying and treating a subject having HCC, more particularly, the early-stage HCC.

One aspect of the present disclosure is directed to a method of identifying and treating a subject having an early-stage hepatocellular carcinoma (HCC). The method comprises:
(a) obtaining a biological sample from the subject,
(b) determining the level of serum amyloid A (SAA) protein in the biological sample; and
(c) providing an anti-cancer treatment to the subject when the level of SAA protein determined in the step (b) is lower than that of a first control sample, or when the level of SAA protein determined in the step (b) is higher than that of a second control sample.

The present method begins by obtaining a biological sample from the subject, which may be a mammal, for example, a human, a mouse, a rat, a hamster, a guinea pig, a rabbit, a dog, a cat, a cow, a goat, a sheep, a monkey, or a horse. Preferably the subject is a human. Depending on the type of the biological sample intended to be obtained, suitable tool and/or procedures may be performed to isolate the biological sample from the subject. Examples of the biological sample suitable for use in the present method may be a whole blood sample, a serum sample, a plasma sample, a urine sample, a saliva sample, a cerebrospinal fluid sample, a synovial fluid sample, a pleural fluid sample, a peritoneal fluid sample, a lymph sample, or a combination thereof. In one working example, the biological sample is a serum sample.

Then, the level of SAA protein in the biological sample (e.g., a serum sample) is determined (step (b)). More specifically, in some embodiments, the level of acute phase SAA (A-SAA) protein is determined. Protein level may be determined by methods well known in the art, which include but are not limited to, gel electrophoresis (e.g., western blot), isotope labeling (e.g., isotope-coded affinity tag (ICAT)), mass spectrometry (MS) (e.g., LC-MS, MALDI-TOF, etc.), enzyme-linked immunosorbent assay (ELISA), immunohistochemistry (IHC), and etc. In some embodiments, the SAA protein level is determined by western blot. In other embodiments, the level of SAA protein is determined by ELISA. In still other embodiments, IHC is used. According to some embodiments of the present disclosure, the SAA protein level thus determined is the sum of its monomers and oligomers. In some preferred embodiments, the SAA protein level refers to the level of its monomers.

Additionally or optionally, prior to step (b), the biological sample is subjected to an ultrasonic treatment, which enhances conversion of normal proteins into abnormal misfolded proteins thereby amplifying the subsequent determined protein level. For better results, the ultrasonic treatment, which is termed “protein misfolding cyclic amplification (PMCA)”, may be repeated, such as 2, 3, or 4 times. In some embodiments, before the step (b), the biological sample containing the monomeric SAA, oligomeric SAA or a combination thereof is subjected to an ultrasonic treatment.

Alternatively or optionally, to minimize variation from one determination to another, the determined level of SAA protein is normalized. The term “normalize or normalization” when applies to a protein refers to the normalized level of a protein product, such as the normalized value determined for the amount of aggregated protein or the amount of amyloid fibrils which might be resulted from ultrasonic treatment, or for the level of indicators (e.g., a fluorescent protein) that bound to the aggregated proteins. In some embodiments, the indicator is Thioflavin T (ThT), which emits fluorescent light that can be measured by a conventional microplate reader. In working examples of the present disclosure, the ThT signal is measured and normalized after amplification of misfolded proteins via PMCA method; and the normalized signal represents the level of SAA protein in the biological sample.

Once the level of SAA protein in the biological sample is determined and optionally normalized, it may then be used as an indicator for the determination of whether an anti-cancer treatment should be administered to the subject. In some embodiments, when the level of SAA protein determined in the step (b) is lower than that of a first control sample, which is derived from a first control subject having a late stage HCC; then the subject of the biological sample is likely or having the risk of developing an early-stage hepatocellular carcinoma (HCC), thus an anti-cancer treatment is administered to the subject to prevent or ameliorate symptoms associated with the early-stage HCC. In other embodiments, when the level of SAA protein determined in the step (b) is higher than that of a second control sample (i.e., the step (c)), which is derived from a second control subject, who has a liver disease that is any of hepatitis, liver cirrhosis, or a combination thereof; then the subject of the biological sample is likely or having the risk of developing an early-stage HCC, thus an anti-cancer treatment is administered to the subject to prevent or ameliorate symptoms associated with the early-stage HCC.

According to some embodiments of the present disclosure, the level of monomeric SAA protein in the first control sample, which is derived from late stage HCC patient, is at least 0.9, 1.0, 1.1, 1.2, 1.3, 1.4, or 1.5 ng. In one specific example, the amount of monomeric SAA protein in the first control sample is at least 0.9 ng.

According to other embodiments of the present disclosure, the level of SAA protein in the second control sample (i.e., the sample derived from a subject having a liver disease such as hepatitis, liver cirrhosis, or etc) is no more than 180, 185, 190, 195, 200, 205, or 210 ng/ml. In one working example, the level of SAA protein in the second control sample is no more than 180 ng/mL. In other embodiments, the amount of monomeric SAA protein in the second control sample is no more than 0.9, 1.0, 1.1, or 1.2 ng. In one specific example, the amount of monomeric SAA protein is no more than 0.9 ng.

Examples of anti-cancer treatments suitable for use in the present method (i.e., for administering to a subject whose SAA protein level meets the required level described above) include, but are not limited to, surgery, radiofrequency ablation, laser ablation, microwave ablation, systemic chemotherapy, percutaneous alcohol injection, percutaneous acetic acid injection, transarterial chemoembolization, adjuvant immunotherapy, adjuvant antiviral treatment, orthotopic liver transplantation, and a combination thereof. Any clinical artisans may choose a suitable treatment for use in the present method based on factors such as the particular condition being treated, the severity of the condition, the individual patient parameters (including age, physical condition, size, gender and weight), the duration of the treatment, the nature of concurrent therapy (if any), the specific route of administration and like factors within the knowledge and expertise of the health practitioner.

Additionally or alternatively, in the present method, the subject providing the biological sample of the step (a), the subject providing the first control sample, or the subject providing the second control samples has a serum alpha-fetoprotein (AFP) level lower than 10 ng/mL.

Also disclosed herein is a method of diagnosing whether a subject has an early-stage HCC via use of a biological sample isolated from the subject. The method comprises determining the level of serum amyloid A (SAA) protein in the isolated biological sample, in which a difference between the determined level of SAA of the isolated biological sample and that of a control sample indicates that the subject has the early-stage HCC; and the control sample is derived from a subject having a liver disease that is any of a late stage HCC, hepatitis, cirrhosis, or a combination thereof.

According to the present disclosure, the SAA level in the isolated biological sample may be determined according to methods described above, and examples of biological sample are substantially same as those described above, therefore description to these steps are omitted herein for the sake of brevity.

In the present method, whether the subject has an early-stage HCC is diagnosed based on the determined level of SAA protein in the isolated biological sample. To this purpose, the determined level of SAA protein in the biological sample is compared with that of a control sample, which may be derived from late stage HCC patients or patients with a liver disease, such as hepatitis, cirrhotic liver, or etc. In the case when the determined level of SAA of the isolated biological sample differs from that of the control sample, such as when the determined level of SAA protein is higher or lower than that of the control sample, then the subject likely or having the risk of developing an early-stage HCC. Accordingly, a suitable treatment is administered to the subject to prevent or ameliorate symptoms associated with the early-stage HCC.

By the virtue of the above features, the present method can provide early identification and detection of early-stage HCC, even if the subject has a low AFP level. According to some embodiments of the present disclosure, the subject providing the biological sample or the subject who provides the control sample has a serum alpha-fetoprotein (AFP) level lower than 10 ng/mL. In some preferred embodiments, the AFP level in the subject is lower than 7 ng/mL. In addition, the detection of monomeric SAA proteins may differentiate HCC patients from other liver diseases, e.g., hepatitis and/or liver cirrhosis, thereby allowing the identified patients to be treated properly.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Examples

Materials and Methods

Subjects and Patients

The patients selected from National Taiwan University Hospital (NTUH) with chronic liver diseases were followed regularly in the liver clinics. The study population were enrolled and categorized into three individual cohorts. hepatitis (H), liver cirrhosis (LC), and hepatocellular carcinoma (HCC) according to the actual liver diseases. The patients with available sera were included in the study consecutively. Total acute-phase SAA (hereinafter, A-SAA) concentration in serum was measured using a specified ELISA kit. A-SAA monomer and oligomer were separated by sodium dodecyl sulphate-polyacrylamide gel electrophoresis (SDS-PAGE) and recognized by an anti A-SAA antibody (Abcam, Cambridge, USA). All serum samples after processing were stored at −80° C. until further use. LC and HCC were diagnosed according to clinical specialty, including the guideline published by American Association for the Study of Liver Diseases (AASLD). The liver function tests and alpha-fetoprotein (AFP) level were measured in a Collage of American Pathologists and ISO15189 certified clinical laboratory in the Department of Laboratory Medicine in NTUH. The study conformed to the ethical guidelines of the 1975 Declaration of Helsinki and was approved by the Institutional Review Boards of NTUH (201108073RC and 201207048RIB). All patients provided written informed consents before enrollment.

Enzyme-Linked Immunosorbent Assay (ELISA)

The assessment of acute-phase SAA (A-SAA) protein concentration in the sera from hepatitis, liver cirrhosis, and HCC patients was conducted by using ELISA kit specified for SAA protein (Abcam, Cambridge, USA). Diluted serum sample (100 μL of 20×PBS diluted) was added into the antibody-pre-coated ELISA plate and incubated at 4° C. overnight. Next, biotinylated SAA detection antibody (100 μL) was applied to the plate and recognized by addition of 100 μL of HRP-Streptavidin. Chromogenic substrate was added and the absorbance at 450 nm was then measured using microplate reader (Molecular Devices, San Jose, USA).

Western Blot

The proteins in 20×PBS-diluted serum samples were separated by 8-16% gradient SDS-PAGE precast gels (Bio-Rad, Hercules, USA). For SDS-PGAE, 10 μl of each sample (total 12 μl, which was composed of 9 μl of 20×PBS-diluted serum and 3 μl of 4×sample buffer) was loaded into each well. To preserve oligomeric states of A-SAA during SDS-PAGE, the sample buffer did not contain β-mercaptoethanol and samples were not boiled. The gel was transferred to a polyvinylidene fluoride (PVDF) membrane (250 mA, 75 min). A-SAA proteins were then recognized by an anti A-SAA antibody (Abcam, Cambridge, USA) in a 1:4,000 dilution ratio and a secondary antibody (goat anti-mouse IgG peroxidase conjugated antibody, 1:10,000) and visualized in a luminescence/fluorescence imaging system (GE Healthcare Life Science, Marlborough, USA). The amount of monomeric A-SAA and its high molecular weight oligomers was quantified by an image analysis system (GE Healthcare Life Science, Marlborough, USA).

After image quantification, the acquired value of A-SAA monomer can be further converted into weight through the following equations:
1. Total A-SAA weight per lane (ng)=total A-SAA concentration (ng/mL)×9/1000×10/12; and
2. Weight of A-SAA monomer (ng)=total A-SAA weight per lane×(image quantification value of monomer)/(image quantification value of monomer+image quantification value of oligomer).

Expression and Purification of Recombinant Human 4SAA1

Full-length human SAA1 (residues 1-104) was cloned into a pET-14b vector which contains an N-terminal histidine tag (His-tag) and expressed in E. coli BL21 cells. A TEV protease cleavage site (ENLYFQS) was added to the N-terminus of mature SAA1 sequence for His-tag removal. Since TEV protease recognizes and cleaves between Q and S, the SAA1 after cleavage contains an additional serine residue at the N-terminus. The overexpression of His-tagged SAA1 protein was induced at 37° C. by the addition of 1 mM IPTG at mid-exponential growth phase of E. coli. After 5-hr induction, the bacteria were harvested and lysed in lysis buffer (20 mM Tris, pH 8.0, 5 M urea, 50 mM imidazole) by a microfluidizer (Microfluidics, Westwood, USA). His-tagged SAA1 protein was purified by a chromatography column (GE Healthcare, Marlborough, USA) in the presence of 5 M urea. The His-tag was removed from SAA1 by incubation of His-tagged TEV protease in a TEV:SAA molar ratio of 1:20 at 30° C. for 16 hr. The cleaved protein was further purified by HisTrap FF column as flow-through. The pure SAA1 protein was then dialyzed into a fresh 50 mM phosphate buffer (pH 7.0) and stored at −80° C. for subsequent experiments.

Protein Misfolding Cyclic Amplification (PMCA)

PMCA reactions were conducted by referring current knowledge (Moda et al., 2014) with some modifications. The sera of patients from three cohorts and purified recombinant human SAA1 were used as seeds and substrates, respectively. As a substrate, SAA1 with a final concentration of 20 μM was prepared in a 50 mM phosphate buffer (pH 7.0). Patients' sera were mixed with purified SAA1 in a volume ratio of 1:20, and then added with a final concentration 10 μM of Thioflavin (ThT) to monitor amyloid fibril formation of SAA1. SAA1-serum sample (100 μL) was loaded into a 96-well ELISA plate and PMCA reaction was automatically performed by a microsonicator (Qsonica, Newtown, USA). One PMCA cycle comprised 29 min 20 sec of incubation at 25° C., followed by a 40-second sonication pulse at a potency of 300 to 310 W. A complete PMCA round contained 24 cycles that equals to 12 hr. The fluorescent emission of ThT was measured at 485 nm using microplate reader (Molecular Devices, San Jose, USA) with an excitation wavelength of 442 nm. The final result for each sample was calculated by normalization of the post-PMCA ThT signal to the mean value of ThT signal obtained from the hepatitis group.

Statistical Analysis

All the statistical analyses in this study were performed and graphed by utilizing analysis software (GraphPad Software version 7.04 and 8.4.2, California, USA). One-way ANOVA was used to evaluate the difference of parameters for more than two cohorts. Post-hoc analyses were conducted by Tukey's multiple comparison between two groups (hepatitis (H) vs. LC, hepatitis vs. HCC, and LC vs. HCC). Correlation between two independent variables was measured by the Spearman's correlation coefficient. Area under the receiver operating characteristic (AUROC) curves were used to assess the predictive capacity of each biomarker, and the Youden index was used to indicate the optimal threshold for each AUROC curve. Multivariable logistic regression was used to evaluate the association of biomarkers with the outcome under adjustment of age, gender, and AST/ALT concentration. In all the analyses, a p-value (two-tailed) less than 0.05 was considered a statistical significance.

### Example 1 Study of Total A-SAA Concentration in Hepatitis, LC and HCC Patients

In this example, the level of A-SAA proteins in serum collected from three cohorts was determined by ELISA. A total of 166 serum samples were tested, which include 40 hepatitis carriers, 30 LC patients, and 96 HCC patients. The demographic and clinical characteristics of the three cohorts are listed in Table 1.

TABLE 1
Characteristics of study participants

| | HCC | Hepatitis | LC | |
|---|---|---|---|---|
| | (n = 96) | (n = 40) | (n = 30) | p-value |
| Age, mean ± SD, years | 60 ± 11 | 47 ± 16 | 60 ± 10 | <.0001 |
| Gender, n (%) | | | | |
| Male | 69 (71.9%) | 17 (42.5%) | 19 (63.3%) | 0.0053 |
| Female | 27 (28.1%) | 23 (57.5%) | 11 (36.7%) | |
| Laboratory test | | | | |
| AST (U/L) | 80 ± 88 | 35 ± 41 | 37 ± 27 | .001 |
| ALT(U/L) | 57 ± 51 | 47 ± 78 | 35 ± 29 | .1475 |
| Cirrhosis, n (%) | 86 (90%) | NA | 30 (100%) | |
| AFP level, n (%) | | | | |
| cut-off: <10 ng/mL | 24 (25%) | 30 (75%) | 27 (90%) | |
| cut-off: <7 ng/mL | 19 (20%) | 30 (75%) | 26 (87%) | |
| Tumor stage | | NA | NA | NA |
| BCLC stage 0 (Very early) | 11 (11%) | | | |
| BCLC stage A (Early) | 31 (32%) | | | |
| BCLC stage B (Intermediate) | 28 (29%) | | | |
| BCLC stage C (Advanced) | 21 (22%) | | | |
| BCLC stage D (Terminal) | 5 (5%) | | | |

** Abbreviations: ALT, alanine aminotransferase; AST, aspartate aminotransferase; BCLC, Barcelona Clinic Liver Cancer; HCC, hepatocellular carcinoma; LC, liver cirrhosis; NA, not applicable.

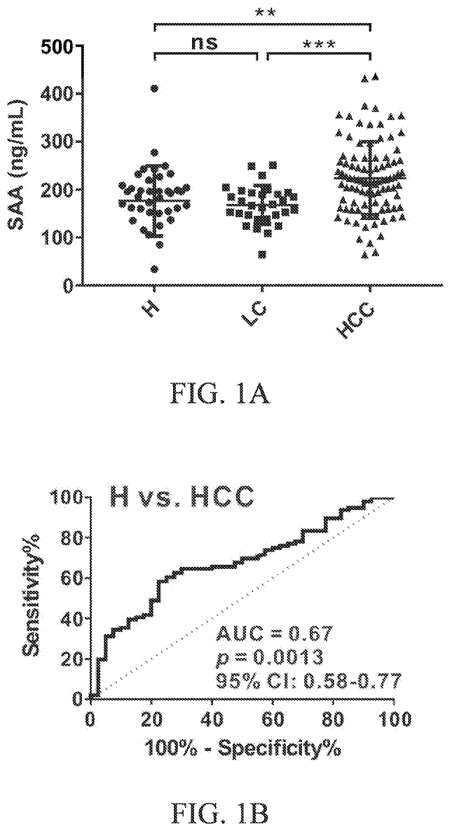

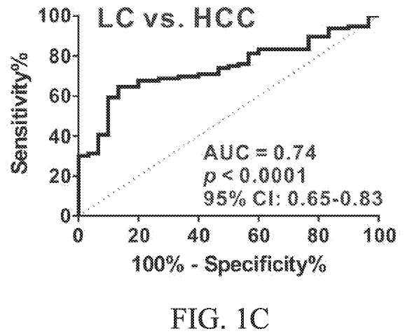

Among the three cohorts, mean concentration of total A-SAA in HCC patients (223.8±75.95 ng/mL) was significantly higher than hepatitis carriers (176.3±73.23 ng/mL) and LC patients (167.8±40.6 ng/mL) with a p-value of 0.0013 and 0.0006, respectively, while no significant difference was found in A-SAA concentration between hepatitis and LC cohorts (FIG. 1A). AUROC curves were generated to assess the diagnostic value of serum A-SAA level to distinguish HCC from hepatitis and LC. The serum A-SAA level showed an area under the AUROC curve of 0.67 for detecting HCC from hepatitis (p=0.0013) (FIG. 1B) and 0.74 to discriminate between LC and HCC (p<0.0001) (FIG. 1C).

### Example 2 A-SAA Monomer Differentiates Hepatitis and LC from HCC

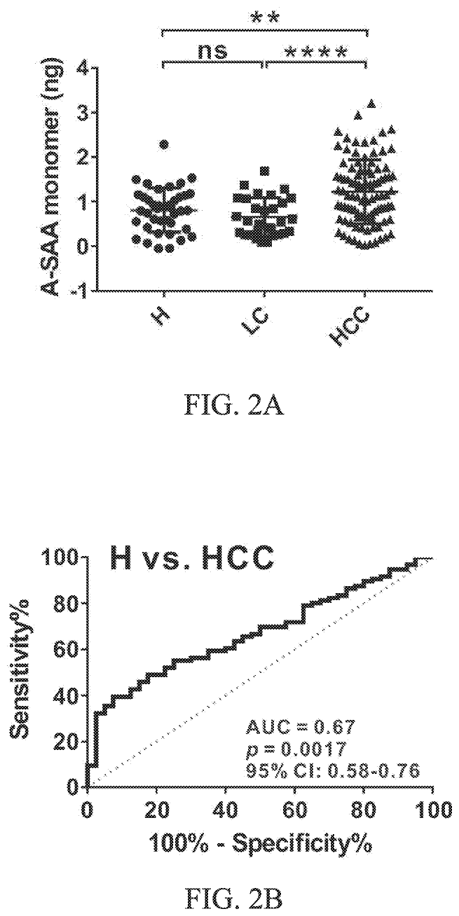

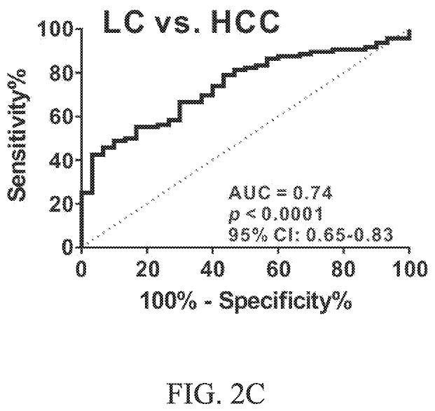

Two main assemblies of A-SAA proteins were discovered in patients' sera: monomeric A-SAA migrated at ˜9 kDa and its high-molecular-weight (HMW) oligomers at ˜130 kDa by using Western blot. The amount of monomeric and oligomeric A-SAA from Western blot results was quantified using analysis program as set forth in “Materials and Methods” section. To observe the possible association between different A-SAA assemblies in the three cohorts, the amount of both monomer and oligomers of A-SAA among three cohorts were further examined. After converted into weight, the weights of monomeric A-SAA was significantly higher in HCC cohort (Mean quantified amount f SD, 1.224±0.7214 ng) when compared to hepatitis (0.6559±0.4305 ng) and LC cohorts (0.8076±0.4949 ng) with a p-value of 0.0016 and <0.0001, respectively (FIG. 2A). No significant difference was found in monomeric A-SAA level between hepatitis and LC cohorts (FIG. 2A). The result is consistent with the ELISA result. In AUROC analysis, monomeric A-SAA level showed an AUC of 0.67 and 0.74 to discriminate between HCC and hepatitis (p=0.0017), and HCC and LC (p<0.0001), respectively (FIGS. 2B and 2C). On the contrary, the Western blot result demonstrated a higher A-SAA oligomer amount in HCC cohort (8290±6467) only when compared to hepatitis cohort (5301±3315; p=0.0125), but not to LC cohort (6970±4397; p=0.4886). Further, the AUROC curves of hepatitis (H) vs. HCC and LC vs. HCC demonstrated the insignificant discrimination (p=0.0213 and p=0.5824, respectively) of A-SAA oligomer (data not shown). Together, the data collectively indicated that HCC subjects may have significant association with monomeric A-SAA but not A-SAA oligomer.

### Example 3 A-SAA Monomer Level Correlates with HCC Staging

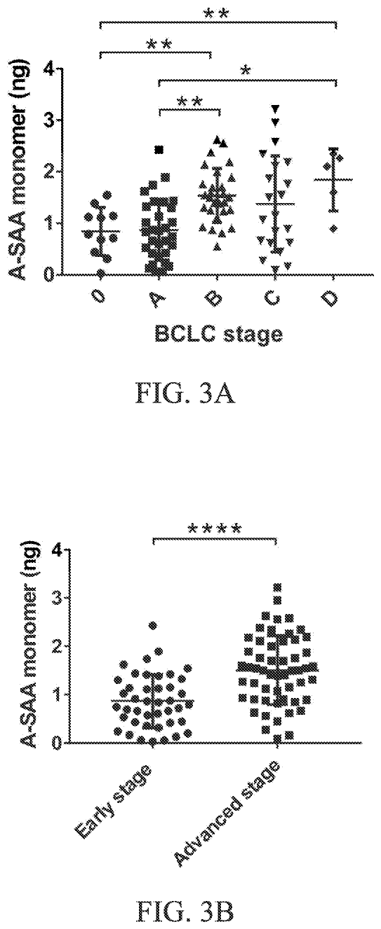

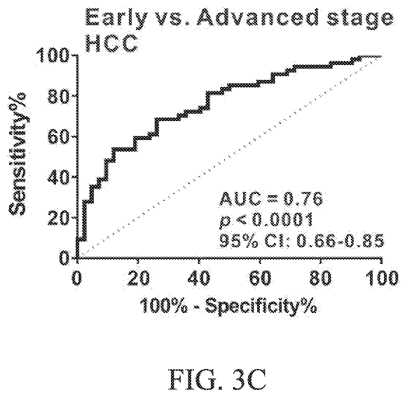

In this example, A-SAA monomer level in different BCLC stages of HCC were further examined. In the analysis between monomeric A-SAA and HCC BCLC stages, we found a significant increase of monomeric A-SAA in HCC stage B and stage D when compared to HCC stage 0 with a p-value of 0.0287 and 0.0444, respectively (FIG. 3A). A significant higher A-SAA monomer level was also observed in HCC stages B and D when compared to stage A with a p-value of 0.0016 and 0.0227, respectively (FIG. 3A). As comparison, no significant difference in monomeric A-SAA level was found in stages C and D when compared to stage B (FIG. 3A). Further, a significant higher A-SAA monomer level was observed in advanced stage (i.e. the combination of stages B, C, and D) when compared to early stages (i.e. the combination of stages 0 and A) with a p-value<0.0001 as depicted in FIG. 3B. The results depicted in FIGS. 3A and 3B indicated that monomeric A-SAA is significantly lower in early stages (stage 0 and A) when compared to more advanced stages (stage B, C, and D). AUROC curve of monomeric A-SAA for discrimination between early and more advanced stage further showed an AUC of 0.76 with a p-value<0.0001 (FIG. 3C). These results showed that level of A-SAA monomer has trends to increase when HCC is progressed from early stages to more advanced stages. The data collectively suggested that monomeric A-SAA may serve as an indicator for the progression of the disease.

### Example 4 the Recombinant Full-Length Human SAA has Excellent Potential to Differentiate LC from Early-Stage HCC

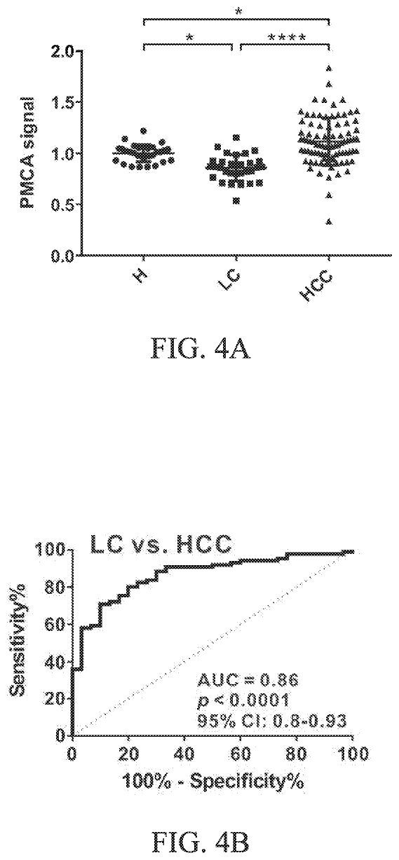

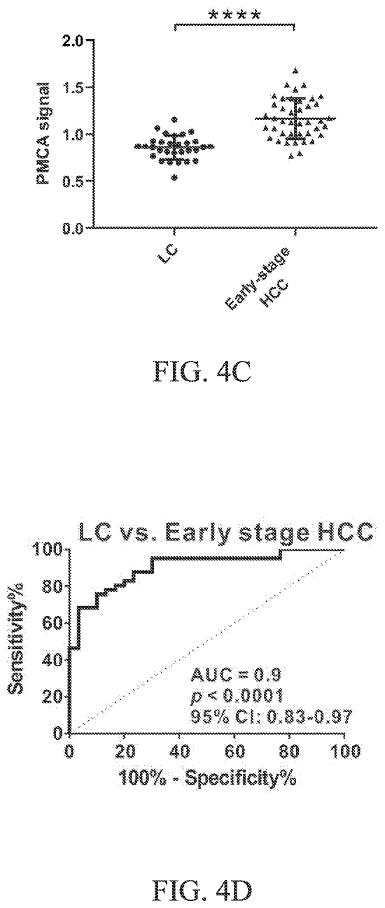

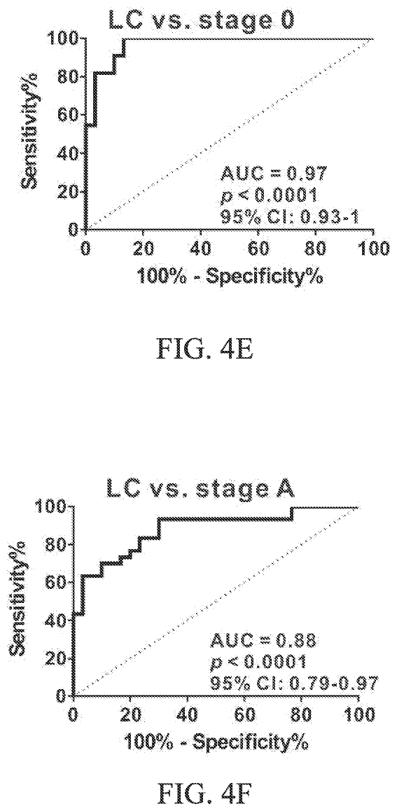

To examine the potential of A-SAA as a diagnostic indicator for HCC, protein misfolding cyclic amplification (PMCA) was applied on A-SAA protein because A-SAA protein possesses the amyloidogenic property. In this example, the purified recombinant full-length human SAA1 was used as a substrate and serum from each patient was used as seeds for PMCA in accordance with the protocol described in “Materials and Methods” section. The results depicted in FIG. 4A indicated that the normalized PMCA signals of HCC patients (1.12±0.23) were significantly higher than that of hepatitis carriers (1±0.08) and LC patients (0.86±0.13) with a p-value of 0.0129 and <0.0001, respectively. Significant difference also showed between hepatitis and LC cohorts (p=0.0123, FIG. 4A). The AUROC curves of the PMCA method showed better AUC of 0.86 to distinguish HCC from LC (p<0.0001), respectively, (FIG. 4B). The capacity of PMCA to distinguish between LC and early-stage HCC was further evaluated, and the normalized signal of LC group and early-stage HCC (stage 0 and stage A) group were compared. It was found that the signal of early-stage HCC patients (1.17±0.21) was significantly higher than that of LC patients (0.86±0.13) with a p-value<0.0001 (FIG. 4C). The AUROC curve also suggested that (AUC of 0.9 (p<0.0001)(FIG. 4D). The PMCA signals and AUROC curves of LC versus stages 0 and A of HCC were respectively analyzed and depicted in FIG. 4E and FIG. 4F, which indicated that the PMCA method using human full-length SAA1 as target processes a good capacity to discriminate LC and HCC, especially for early stages of HCC.

### Example 5 A-SAA Protein and A-SAA Derived Biomarkers Discriminate HCC in Patients with Low AFP Levels

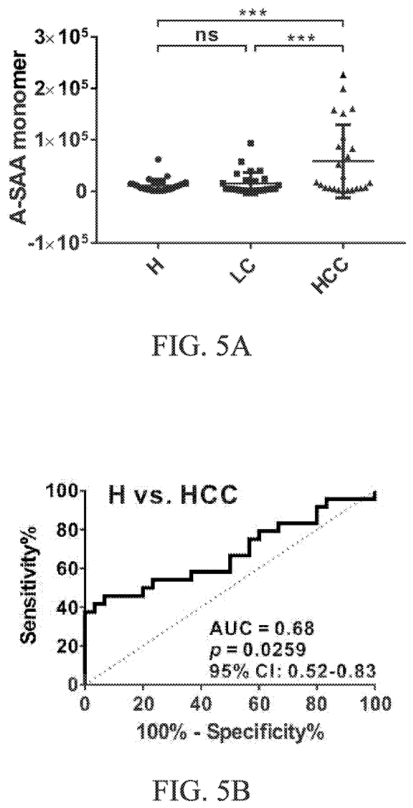

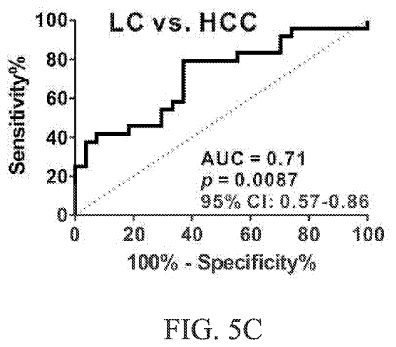

AFP is a commonly-used marker for HCC surveillance. However, as set forth above, still about 20% of advanced HCC patients whose serum AFP level remains normal, resulting inaccurate diagnosis of HCC. In this study, a correlation between the amount of monomeric A-SAA and AFP was analyzed to evaluate whether A-SAA is an independent biomarker to AFP. After excluding the patients whose serum AFP concentration is higher than or equal to 10 ng/mL, a total of 81 serum samples from 30 hepatitis (indicated as H), 27 LC, and 24 HCC patients were analyzed (as listed in Table 1). Since a preliminary analytical result showed a weak correlation between A-SAA monomer and AFP level with a Spearman's coefficient of 0.18 (p=0.0387) (data not shown), therefore, the diagnostic capacity of A-SAA for HCC patients having low AFP level (<10 ng/mL) was further evaluated. The result was depicted in FIG. 5A, indicating that the A-SAA monomer level in HCC (58703±71114) still significantly higher than those in the hepatitis cohort (11198±12149, p=0.0002) and LC cohort (15141±21713, p=0.0009). The AUROC curves showed that AUCs for A-SAA monomer to discriminate HCC from hepatitis and LC, are 0.68 (p=0.0259) and 0.71 (p=0.0087), respectively (FIGS. 5B and 5C).

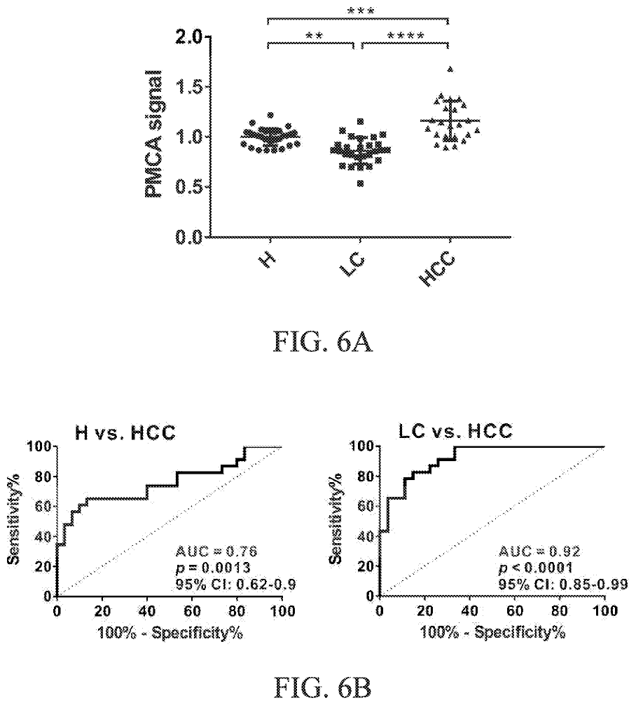

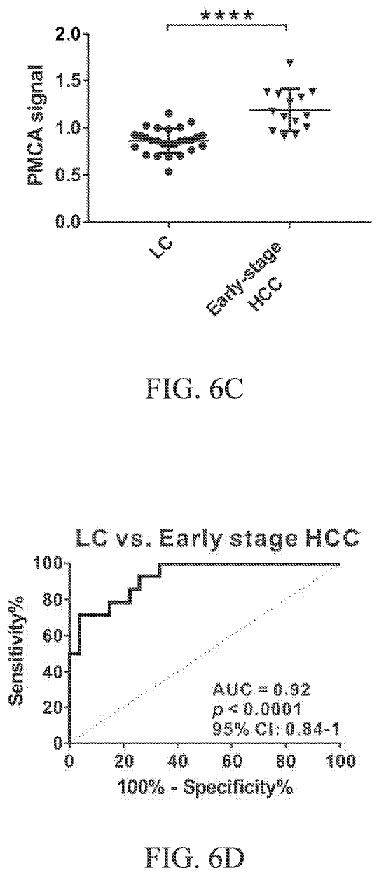

The PMCA analysis shows consistency. In the PMCA analysis, the signals of HCC cases (1.16±0.2) remained significantly higher than that of the hepatitis (1±0.08, p=0.0002) and LC cohorts (0.86±0.13, p<0.0001) in the patients with low AFP level, and the LC cases further showed significantly lower ThT signals than the hepatitis carriers (p=0.0012), referring to FIG. 6A. The AUROC curves of the PMCA method further displayed improved AUCs of 0.76 (p=0.0013) and 0.92 (p<0.0001) for hepatitis (H) vs. HCC and LC vs. HCC, respectively (FIG. 6B). When comparing LC and the early-stage HCC, the PMCA signal of LC patients (0.86±0.13) remained significantly lower than that of the early-stage HCC patients with low AFP level (1.19±0.22) with a p-value<0.0001 (FIG. 6C). The AUROC analysis showed a high AUC of 0.92 (p<0.0001) for discrimination between LC and early-stage of HCC (FIG. 6D). Those results suggested that SAA proteins combined with the PMCA method possesses a great potential to aid the early diagnosis of HCC (e.g., stage 0 and/or stage A) especially in patients with low AFP level.

### Example 6 A-SAA Derived Biomarkers Discriminate HCC in Cirrhotic Patients

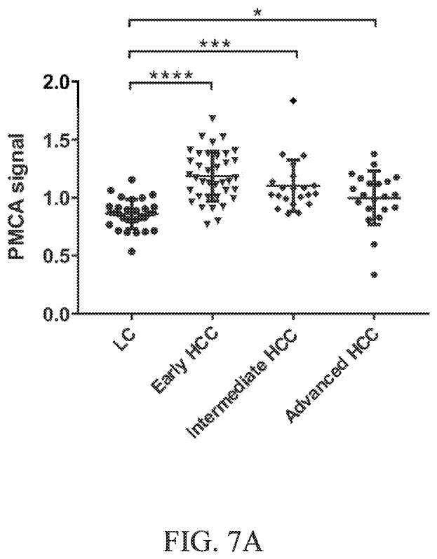

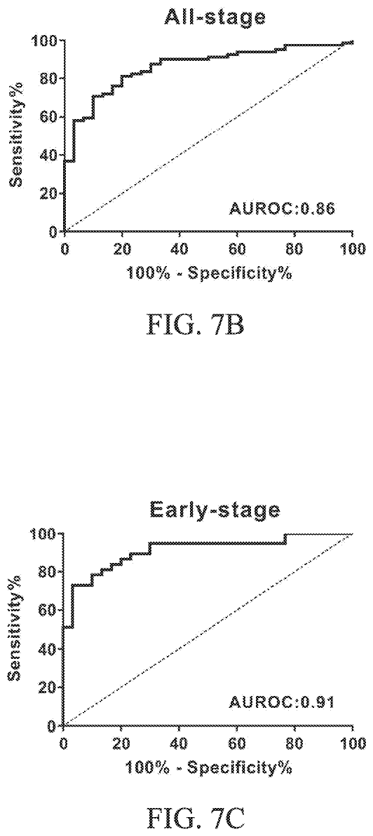

In general, patients who suffer from a cirrhotic liver are diagnosed LC and having higher risk of HCC. In this study, whether the A-SAA derived biomarkers (i.e., PMCA signals) are able to discriminate HCC in cirrhotic patients either with or without low AFP were verified by analyzing 116 cirrhotic patients after excluding 50 non-cirrhotic ones (Table 1). Data depicted in FIG. 7A indicated that, when compared to all BCLC stages of HCC, the PMCA signals of LC patients were significantly lower than those of all-stage HCC groups. Specifically, PMCA signals were 1.19±0.21 in early-stage HCC, while LC patients showed 0.86±0.13 of PMCA signals. The ROC analysis provided in FIGS. 7B and 7C further indicated high AUROCs of 0.86 (p<0.0001) and 0.91 (p<0.0001) in all-stage HCC (FIG. 7B) and early-stage HCC (FIG. 7C) among patients that possess cirrhotic livers. The data is a clear indication that the PMCA signals are able to differentiate general liver cirrhosis and HCC patients, particularly HCC patient in its early-stage.

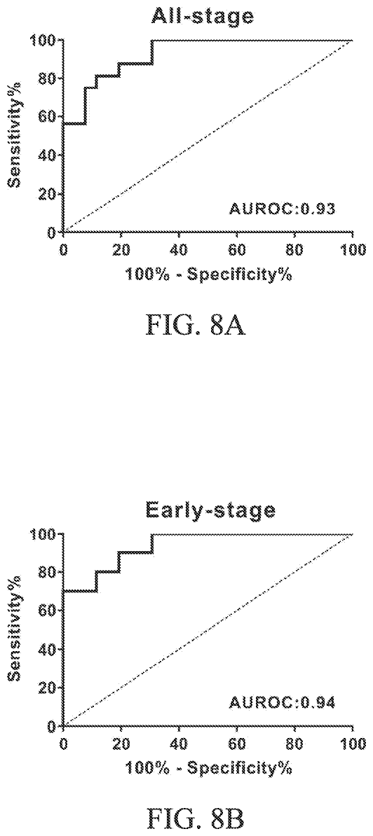

Further, the sensitivity and specificity of PMCA signals were examined to evaluate whether PMCA signal may serve as a reliable indicator for detecting early HCC in cirrhotic patients in the case of low AFP level. To this purpose, only 17 cirrhotic patients with low AFP level (<7 ng/mL) were included and examined in this experiment. Results are summarized in Table 2 and depicted in FIGS. 8A-8B. The data collectively demonstrated a high sensitivity and specificity for PMCA for all-stage HCC detection and early-stage HCC detection in cirrhotic patients with low AFP level. Specifically, for the early-stage HCC detection, the AUROC of PMCA reached 0.94 (P<0.0001) with a sensitivity of 90% and specificity of 81%, indicating that the PMCA signals demonstrate a better performance in detecting early-stage of HCC among cirrhotic patients with advanced sensitivity and specificity than AFP.

TABLE 2
Performance of PMCA signals for detection of all-stage
and early-stage HCC in cirrhotic patients with low AFP

| | AUROC | | Sensitivity | Specificity |
|---|---|---|---|---|
| | (95% CI) | P-value | (%) (95% CI) | (%) (95% CI) |
| All-stage | 0.93 (0.85-1) | <0.0001 | 81 (54-96) | 88 (70-98) |
| (Cirrhotic + | | | | |
| low AFP) | | | | |
| n = 17 | | | | |
| Early-stage | 0.94 (0.86-1) | <0.0001 | 90 (56-100) | 81 (61-93) |
| (Cirrhotic + | | | | |
| low AFP) | | | | |
| n = 10 | | | | |

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method of identifying and treating a subject having an early-stage hepatocellular carcinoma (HCC), comprising,
(a) obtaining a biological sample from the subject;
(b) determining the level of serum amyloid A (SAA) protein in the biological sample; and
(c) providing an anti-cancer treatment to the subject when the determined level of SAA protein is lower than that of a first control sample or higher than that of a second control sample;
wherein,
the anticancer treatment is selected from the group consisting of a surgery, a radiofrequency ablation, a laser ablation, a microwave ablation, a systemic chemotherapy, a percutaneous alcohol injection, a percutaneous acetic acid injection, a transarterial chemoembolization, an adjuvant immunotherapy, an adjuvant antiviral treatment, an orthotopic liver transplantation, and a combination thereof;
the first control sample is derived from a subject having a late stage HCC; and
the second control sample is derived from a subject having a liver disease that is any of hepatitis, cirrhosis, or a combination thereof.

2. The method of claim 1, wherein the SAA protein is in monomeric form.

3. The method of claim 1, wherein the level of SAA protein in the first control sample is at least 0.9 ng, and the level of SAA protein in the second control sample is no greater than 0.9 ng, or no greater than 180 ng/mL.

4. The method of claim 1, wherein the subject has a serum alpha-fetoprotein (AFP) level lower than 10 ng/mL.

5. The method of claim 1, wherein the biological sample is a whole blood sample, a serum sample, a plasma sample, a urine sample, a saliva sample, a cerebrospinal fluid sample, a synovial fluid sample, a pleural fluid sample, a peritoneal fluid sample, a lymph sample, or a combination thereof.

6. A method of identifying and treating a subject having an early-stage hepatocellular carcinoma (HCC), comprising,
(a) obtaining a biological sample from the subject;
(b) subjecting the biological sample to an ultrasonic treatment;
(c) determining the level of serum amyloid A (SAA) protein in the ultrasonic treated biological sample of step (b); and
(d) providing an anti-cancer treatment to the subject when the determined level of SAA protein is higher than that of a control sample,
wherein,
the anticancer treatment is selected from the group consisting of a surgery, a radiofrequency ablation, a laser ablation, a microwave ablation, a systemic chemotherapy, a percutaneous alcohol injection, a percutaneous acetic acid injection, a transarterial chemoembolization, an adjuvant immunotherapy, an adjuvant antiviral treatment, an orthotopic liver transplantation, and a combination thereof; and
the control sample is derived from a subject having a liver disease that is any of a late stage HCC, hepatitis, cirrhosis, or a combination thereof.

7. The method of claim 6, wherein the subject has a serum alpha-fetoprotein (AFP) level lower than 10 ng/mL.

8. The method of claim 7, wherein the subject has the AFP level lower than 7 ng/mL.

9. The method of claim 6, wherein the biological sample is a whole blood sample, a serum sample, a plasma sample, a urine sample, a saliva sample, a cerebrospinal fluid sample, a synovial fluid sample, a pleural fluid sample, a peritoneal fluid sample, a lymph sample, or a combination thereof.

10-15. (canceled)


SEQUENCE LISTING (1 sequence)

SEQ ID NO: 1 - protein, 7 aa, Artificial Sequence
  TEV protease cleavage site
ENLYFQS